(19) United States
(12) Patent Application Publication
(10) Pub. No.: US 2007/0178151 A1
(43) Pub. Date: Aug. 2, 2007

(54) Spray dried pharmaceutical compositions

(76) Inventors: Vlasios Andronis, King of Prussia, PA (US); Rennan Pan, King of Prussia, PA (US); Kamlesh Rameshchandra Patel, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/593,653

(22) PCT Filed: Mar. 28, 2005
(86) PCT No.: PCT/US05/10350

§ 371 Date: Sep. 21, 2006

Related U.S. Application Data
(60) Provisional application No. 60/557,571, filed on Mar. 30, 2004.

(51) Int. Cl. A61K 9/20; B29B 9/00

(52) U.S. Cl. 424/464; 264/5

(57) ABSTRACT

The present invention relates to novel compositions containing the NK₃ receptor antagonist talnetant which compositions have enhanced bioavailability. In addition, the invention relates to processes for the preparation and to uses of the compositions in therapy.

The present invention relates to novel compositions containing the NK3 receptor antagonist talnetant [(S)-(−)-N-(α-ethylbenzyl)-3-hydroxy-2-phenylquinoline4-carboxamide] which compositions have enhanced bioavailability. In addition, the invention relates to processes for the preparation and to uses of the compositions in therapy.

Talnetant, its preparation and its use in the treatment of pulmonary disorders, disorders of the central nervous system and neurodegenerative disorders are disclosed in published International Patent application WO 95/32948. Published International Patent applications WO 97/19927, WO 97/19928, WO 99/14196 and WO 02/094187 disclose additional therapeutic utilities for talnetant, pharmaceutically acceptable salts and processes for its preparation. The above-mentioned patent applications are herein incorporated by reference as if each individual publication were specifically and individually indicated to be incorporated by reference herein as though fully set forth.

Talnetant has low aqueous solubility (approximately 0.03 mg/mL at pH 1 and 0.001 mg/ml, at pH 7.0). Typically drugs with low aqueous solubility are absorbed slowly across the walls of the gastrointestinal tract (GIT) due to poor dissolution of the solid in the GIT leading to a small diffusive driving force.

There are a number of different methods employed to improve absorption of a particular drug substance. It may be possible to develop so-called prodrugs or salts of the active agent, i.e. more soluble derivatives, by attaching a solubilizing group (e.g. phosphate, succinate or polyethylene glycol) to the drug, thereby taking advantage of the high solubility and dissolution rate of the derivative prodrug/salt. Alternatively, it is known to use physical formulation methods, such as use of amorphous drug or dispersion in a soluble carrier to increase the dissolution rate of the drug product and hence the absorption rate (J. H. Fincher, J. Pharm. Sci., 1968, 57, 1825 and G. L. Amidon et at, J. Pharm. Sci., 1980, 12, 1363).

According to a second aspect, the invention provides a spray dried pharmaceutical composition comprising a) talnetant particles having a Dᵥ90 in the range from 0.1 to 2.0 μm, and b) one or more ionic surfactants.

We have surprisingly discovered that a spray-dried pharmaceutical composition according to the second aspect greatly improves bioavailability.

Preferred ionic surfactants are sodium lauryl sulfate and dioctyl sodium sulfosuccinate (docusate sodium). A particularly preferred surfactant is sodium lauryl sulfate.

Preferably the concentration of surfactant in the spray dried composition is 0.5 to 3.0% by weight of talnetant. Preferably the concentration of surfactant in the dispersion prior to spray drying is 0.05 to 5% by weight of dispersion, more preferably 0.05 to 2%.

Preferably the spray-dried composition comprises one or more anti-agglomeration agents (for example polyvinyl pyrolidone (PVP) or Povidone, hydroxypropyl methyl cellulose and hydroxyethyl cellulose and hydroxypropylcellulose). Preferably the concentration of the anti-aglomeration agent in the spray-dried composition is 2 to 10% by weight of talnetant. Preferably the concentration of anti-aglomeration agent in the dispersion prior to spray drying is 0.1 to 10.0% by weight of dispersion, more preferably 0.5 to 5.0%.

Preferably the spray dried composition comprises one or more carriers (for example mannitol, sorbitol, lactose, lacitol, xylitol and starch). Preferably the concentration of the carrier in the spray dried composition is 10 to 50% by weight of talnetant. Preferably the concentration of the carrier in the dispersion prior to spray drying is 0.1 to 30% by weight of dispersion, more preferably 5 to 15%.

The spray-dried composition and dispersion prior to spray drying may contain further suitable pharmaceutically acceptable excipients. Suitable excipients are described in the Handbook of Pharmaceutical Excipients, Pharmaceutical Press, 1986, published by The American Pharmaceutical Association and The Royal Pharmaceutical Society of Great Britain. Examples of further excipients include stablilisers to maintain the particles in suspension.

The spray dried composition may be administered to the subject without further processing, however it will generally be formulated into other dosage forms in conjunction with further pharmaceutically acceptable excipients selected with regard to the desired dosage form. These further excipients will typically be added to the spray dried composition after spray drying.

Preferably the dosage form is administered orally. Oral administration will typically involve swallowing so that the compound enters the GIT. Dosage forms for oral administration include solid formulations such as tablets, capsules containing particulates or powders, sachets, vials, powders, granules, lozenges, reconstitutable powders and liquid preparations (such as suspensions, emulsions and elixirs).

Oral dosage forms may contain further excipients such as binding agents (for example syrup, acacia, gelatin, sorbitol, starch, PVP, HPMC, and tragacanth); fillers (for example lactose, sugar, maize-starch, calcium phosphate, sorbitol and glycine); tabletting lubricants (for example magnesium stearate); and disintegrants (for example starch, sodium starch glycollate and microcrystalline cellulose). In addition, the oral dosage form may contain preservatives, anti-oxidant, flavours, granulation binders, wetting agents and colourants.

Preferably the dosage form for oral administration is a tablet. Tablets may be prepared using standard technology familiar to the formulation chemist, for example by direct compression, granulation, melt congealing and extrusion. The tablet may be coated or uncoated. The tablet may be formulated to be immediate or controlled release. Controlled release formulations include delayed-, sustained-, pulsed or dual-release. Suitable tabletting excipients are described in the Handbook of Pharmaceutical Excipients, Pharmaceutical Press, 1986, published by The American Pharmaceutical Association and The Royal Pharmaceutical Society of Great Britain. Typical tabletting excipients include: carriers (for example lactose and starch), lubricating agents (for example magnesium stearate), binding agents, wetting agents, colorants, flavourings, glidants and disintegrants (for example croscarmellose sodium).

The composition of a preferred tablet according to the invention is Tablet A described hereinafter in Example 2.

Excipients suitable for preparing liquid dosage forms include: suspending agents (for example sorbitol, syrup, methyl cellulose, gelatin, hydroxyethylcellulose, carboxymethylcellulose, aluminium stearate gel and hydrogenated edible fats); emulsifying agents (for example lecithin, sorbitan monooleate and acacia); aqueous or non-aqueous vehicles, which include edible oils (for example almond oil and fractionated coconut oil), oily esters (for example esters of glycerine and propylene glycol), ethyl alcohol, glycerine, water and normal saline; preservatives (for example methyl, propyl p-hydroxybenzoate and sorbic acid); and if desired conventional flavouring or colouring agents.

The effective dose of talnetant depends on the condition of the patient, the frequency and route of administration. A unit dose will generally contain from 20 to 1000 mg of talnetant, preferably 30 to 500 mg, most preferably 200 or 400 mg. The unit dose may be administered one or more times per day (for example 2; 3 or 4 times per day). The total daily dose for a 70 kg adult will normally be in the range 100 to 3000 mg. Alternatively the unit dose will contain from 2 to 20 mg of active ingredient and be administered in multiples, if desired, to give the preceding daily dose.

The compositions and tablets of the invention are preferably adapted for use in the medical or veterinarial fields. For example, such preparations may be in a pack form accompanied by written or printed instructions for use as an agent In the treatment of the conditions.

NK3 receptor antagonists, including talnetant, are useful in the treatment and prevention of a wide variety of clinical diseases and conditions characterised by overstimulation of the NK3 receptors. These diseases and conditions (hereinafter referred to as “diseases and conditions of the inventions”) include: CNS disorders such as depression (which term Includes bipolar (manic) depression (including type I and type II), unipolar depression, single or recurrent major depressive episodes with or without psychotic features, catatonic features, melancholic features, atypical features (e.g. lethargy, over-eating/obesity, hypersomnia) or postpartum onset, seasonal affective disorder and dysthymia, depression-related anxiety, psychotic depression, and depressive disorders resulting from a general medical condition including, but not limited to, myocardial infarction, diabetes, miscarriage or abortion); anxiety disorders (including generalised anxiety disorder (GAD), social anxiety disorder (SAD), agitation, tension, social or emotional withdrawal in psychotic patients, panic disorder, and obsessive compulsive disorder); phobias (including agoraphobia and social phobia); psychosis and psychotic disorders (including schizophrenia, schizo-affective disorder, schizophreniform diseases, acute psychosis, alcohol psychosis, autism, delerium, mania (including acute mania), manic depressive psychosis, hallucination, endogenous psychosis, organic psychosyndrome, paranoid and delusional disorders, puerperal psychosis, and psychosis associated with neurodegenerative diseases such as Alzheimer's diease); post-traumatic stress disorder; attention deficit hyperactive disorder (ADHD); cognitive impairment (e.g. the treatment of impairment of cognitive functions including attention, orientation, memory (memory disorders, amnesia, amnesic disorders and age-associated memory impairment) and language function, and including cognitive impairment as a result of stroke, Alzheimer's disease, Aids-related dementia or other dementia states, as well as other acute or sub-acute conditions that may cause cognitive decline such as delirium or depression (pseudodementla states)); convulsive disorders such as epilepsy (which includes simple partial seizures, complex partial seizures, secondary generalised seizures, generalised seizures Including absence seizures, myoclonic seizures, clonic seizures, tonic seizures, tonic clonic seizures and atonic seizures); psychosexual dysfunction (including inhibited sexual desire (low libido), inhibited sexual arousal or excitement, orgasm dysfunction, inhibited female orgasm and inhibited male orgasm, hypoactive sexual desire disorder (HSDD), female sexual desire disorder (FSDD), and sexual dysfunction side-effects induced by treatment with antidepressants of the SSRI-class); sleep disorders (including disturbances of circadian rhythm, dyssomnia, insomnia, sleep apnea and narcolepsy); disorders of eating behaviours (including anorexia nervosa and bulimia nervosa); neurodegenerative diseases (such as Alzheimer's disease, ALS, motor neuron disease and other motor disorders such as Parkinson's disease (including relief from locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), dementia in Parkinson's disease, dementia in Huntington's disease, neuroleptic-induced Parkinsonism and tardive dyskinesias, neurodegeneration following stroke, cardiac arrest, pulmonary bypass, traumatic brain injury, spinal cord injury or the like, and demyelinating diseases such as multiple sclerosis and amyotrophic lateral sclerosis); withdrawal from abuse of drugs including smoking cessation or reduction in level or frequency of such activities (such as abuse of cocaine, ethanol, nicotine, benzodiazepines, alcohol, caffeine, phencyclidine and phencyclidine-like compounds, opiates such as cannabis, heroin, morphine, sedative, hypnotic, amphetamine or amphetamine-related drugs such as dextroamphetamine, methylamphetamine or a combination thereof); pain (which includes neuropathic pain (including diabetic neuropathy; sciatica; non-specific lower back pain; multiple sclerosis pain; pain associated with fibromyalgia or cancer; AIDS-related and HIV-related neuropathy; chemotherapy-induced neuropathy; neuralgia, such as post-herpetic neuralgia and trigeminal neuralgia; sympathetically maintained pain and pain resulting from physical trauma, amputation, cancer, toxins or chronic inflammatory conditions such as rheumatoid arthritis and osteoarthritis; reflex sympathetic dystrophy such as shoulder/hand syndrome), acute pain (e.g. musculoskeletal pain, post operative pain and surgical pain), inflammatory pain and chronic pain, pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia), pain associated with migrane, and non-cardiac chest pain); certain CNS-mediated disorders (such as emesis, Irritable bowel syndrome and non-ulcer dyspepsia); and pulmonary disorders (such as asthma, chronic obstructive pulmonary disease (COPD), airway hyperreactivity and cough).

More preferred diseases or conditions (hereinafter referred to as “preferred diseases and conditions of the invention”) mediated by modulation of the NK3 receptor are depression; anxiety disorders; phobias; psychosis and psychotic disorders; post-traumatic stress disorder; attention deficit hyperactive disorder (ADHD); withdrawal from abuse of drugs including smoking cessation or reduction in level or frequency of such activities; irritable bowel syndrome; cognitive impairment; convulsive disorders; psychosexual dysfunction; sleep disorders; disorders of eating behaviours; neurodegenerative diseases; pain; emesis; irritable bowel syndrome; non-ulcer dyspepsia; and pulmonary disorders (such as asthma, chronic obstructive pulmonary disease (COPD), airway hyperreactivity and cough).

No unacceptable toxicological effects were seen when the compositions of the invention were administered.

Suitable milling apparatus for the preparation of a compositions according to the present invention include, conventional wet bead mills such as those manufactured by Nylacast, NETZSCH, DRAIS and others. Suitably, the milling chamber of said milling apparatus is lined with or constructed from an abrasion-resistant polymer material. Preferably, the milling chamber of said milling apparatus is lined with or constructed from nylon. An example of a suitable milling chamber is described in International Patent Application, Publication Number WO 02/00196.

Suitable grinding media for use In the preparation of a pharmaceutical composition according to the present invention Include glass beads and ceramic beads, for example, those made from rare earth oxide materials. The diameter of said grinding media is suitably within the range 0.1 mm to 3 mm, and is preferably within the range 0.3 mm to 0.8 mm. The density of said grinding media is suitably greater than 3 gcm⁻³, and is preferably within the range 5 to 10 gcm⁻³.

Suitable spray drying and spray granulating techniques will be apparent to those skilled in the art (see for example, Gilbert S. Banker, “Modern Pharmaceutics, Drugs and the Pharmaceutical Sciences”, 1996 and references cited therein) and may be effected using a spray dryer, such as the Niro SD 6.3R Spray Dryer, Mobile Minor Niro or a Yamato GA-32 Spray Dryer, or a fluid bed granulator, such as Glatt fluid bed granulator.

Particles prepared according to the present invention may be sized using conventional techniques known in the art, such as laser light diffraction and photon correlation spectroscopy. A suitable particle sizing apparatus is the Malvern Mastersizer. The Malvern Mastersizer and its operation will be familiar to the skilled person with reference to its operating manual.

The following Examples illustrate the present invention.

### EXAMPLE 1

### Preparation of Spray Dried Compositions and Particle-Size Recovery After Dissolution in Water

### a) Composition According to the Invention (Composition 1)

Sodium lauryl sulphate (0.3% w/w), povidone (Kollidone K30) (1.7% w/w) and mannitol powder USP (10.0% w/w) were dissolved in the purified water (68.0% w/w). Solid talnetant (20.0% w/w) was then slowly added with continuous mixing until a homogeneous suspension was obtained. The homogenous suspension was passed through a Netzsch bead mill containing 85% by volume of yttrium-stabilised 0.3 mm zirconium oxide beads. The dispersion was re-circulated with continual mixing until a D_{V}90 of >0.4 μm<1.0 μm was obtained. The D_{V}90 was measured using Malvern Particle Sizer.

This dispersion was spray-dried using a Mobile Minor Niro spray dryer (operated in accordance with the manufacturers instructions) at the following settings: 2 Fluid Nozzle: 2 Bar pressure; Pump Speed: 35 mL/min (suspension spray rate); Inlet Temperature: 150° C.; Outlet Temperature: 60° C.

The spray-dried composition was dispersed in water and the D_{V} was measured using a Malvern Particle Sizer.

### b) Comparative Example (Composition 2)

The procedure and conditions with some modifications to spray drying parameters (outlet temp 110 deg C. and outlet temp 40 deg C.) as described under a) above was repeated except that the ionic surfactant sodium lauryl sulfate was replaced by pluronic F68 (a non-ionic surfactant). The D_{V}90 after milling was the same as for procedure a).

The particle size distribution following redispersion in water for compositions 1 and 2 are shown in Table 1 (The values in parentheses are the corresponding D_{V} values before spray drying). The table shows that composition 1 gives a virtually complete recovery of particle size after redispersion in water, whereas composition 2 results in substantial agglomeration to give much larger particles.

TABLE 1

| | Composition 1 | Composition 2 |
|---|---|---|
| D_{V}10 (μm) | 0.077 (0.080) | 0.13 (0.075) |
| D_{V}50 (μm) | 0.179 (0.190) | 0.55 (0.177) |
| D_{V}90 (μm) | 0.437 (0.488) | 2.75 (0.411) |

### Example 2

### Comparative Study to Evaluate the Effect on pK Parameters of Oral Administration of Compositions of to Conscious Male Beagle Dogs

A catheter was placed in the cephalic vein of each of four fasted male beagle dogs. Each dog was administered half a Tablet A, the composition of which is shown in Table 2 (the composition of the invention). Blood samples were collected via the catheter prior to dosing and at the following times after dosing: 15, 30, 45, 60, 90, 120, 180, 240, 300, 360, 480, 600 and 1440 minutes. Plasma was prepared and stored frozen for analysis. Plasma concentrations of (Limit of quantitation=10 ng/mL) were quantified by an LC/MS/MS method. Noncompartmental analysis was used for pharmacokinetic analysis of plasma concentration versus actual sampling time data. The dogs were fed at 6 hours post-dose (following the collection of the 6-hour blood sample) and food was removed one hour later.

After a wash-out period of one week, the procedure was repeated on the same four dogs with Tablet B. After a further one week wash-out period, the procedure was repeated with Tablet C, and so on with Tablet D. (Tablets B, C and D are comparative examples).

TABLE 2

| Tablet | Constituents (mg/tablet) | Dose (mg/kg) |
|---|---|---|
| A | talnetant (D_{V}90 = 0.5 μm (100) | 5.46 ± 0.60 (n = 3) |
| | Sodium Lauryl Sulfate (1.5) | |
| | Povidone (Kollidone K30) (8.5) | |
| | Mannitol (50) | |
| | Avicel PH 200 (Microcrystalline | |
| | Cellulose) (169.4) | |
| | Crospovidone (38.70) | |
| | Colloidal Silicon Dioxide | |
| | (Cab-O-Sil) (4) | |
| | Magnesium Stearate (2.9) | |
| | Opadry yellow 03B12905 (11.25) | |
| B | talnetant (D_{V}90 = 0.5 μm (100) | 5.09 ± 0.66 (n = 4) |
| | Poloxamer 127 (8.5) | |
| | Povidone (Kollidone K30) (5) | |
| | Natrosol 250SL (HEC) (10) | |
| | Avicel PH 200 (Microcrystalline | |
| | Cellulose) (205.9) | |
| | Crospovidone (38.70) | |
| | Colloidal Silicon Dioxide | |
| | (Cab-O-Sil) (4) | |
| | Magnesium Stearate (2.9) | |
| | Opadry yellow 03B12905 (11.25) | |
| C | Talnetant (D_{V}90 = 0.5 μm (100) | 5.43 ± 0.76 (n = 4) |
| | Poloxamer 188 (20.03) | |
| | Povidone (Kollidone K30) (10.01) | |
| | Avicel PH 200 (Microcrystalline | |
| | Cellulose (199.36) | |
| | Crospovidone (38.70) | |
| | Colloidal Silicon Dioxide | |
| | (Cab-O-Sil) (4) | |
| | Magnesium Stearate (2.9) | |
| | Opadry yellow 03B12905 (11.25) | |
| D | Talnetant (D_{V}90 = 0.5 μm (100) | 5.49 ± 0.68 (n = 4) |
| | Poloxamer 188 (10) | |
| | Povidone (Kollidone K30) (10) | |
| | Avicel PH 200 (Microcrystalline | |
| | Cellulose) (209.4) | |
| | Crospovidone (38.70) | |
| | Colloidal Silicon Dioxide | |
| | (Cab-O-Sil) (4) | |
| | Magnesium Stearate (2.9) | |
| | Opadry yellow 03B12905 (11.25) | |

The results of the study are shown in Table 3. The results show that administration of Tablet A (the composition of the invention) lead to significant improvement in bioavailability compared with the comparative examples (Tablets B, C and D). In addition the results show that the values for Tablet A were the most consistent among the subjects.

The average relative bioavaliability for the three formulations against Tablet C was calculated by determining the relative bioavailibility for each animal then calculating the average and standard deviation of the individual relative bioavailability values. Overall, the average relative bioavailability of Table A was 1.96±0.34, and the individual values were 1.81, 2.35 and 1.72.

Cₘₐₓ is the maximum plasma concentration of achieved. Tₘₐₓ is the time after administration at which the maximum concentration was achieved. AUC is the Area Under the Curve of a plot of plasma concentration against time. AUC(0-t) refers to the area from t=0 to the last quantifiable concentration. AUC (0-360) refers to the area from t=0 to t=360 minutes.

TABLE 3

| | | | AUC | AUC | Relative Oral |
|---|---|---|---|---|---|
| | | | (0-360) | (0-t) | Bioavailability |
| Tab- | Cₘₐₓ | Tₘₐₓ | (min · | (min · | AUC(0-360) |
| let | (μg/mL) | (min) | μg/mL) | μg/mL) | A̅U̅C̅(̅0̅-̅3̅6̅0̅)̅_̅{̅T̅a̅b̅l̅e̅t̅ C̅}̅ |
| A | 4.04 ± | 521.0 ± | 742.1 ± | 3306.0 ± | 1.96 ± 0.34 |
| | 0.287 | 68.4 | 55.0 | 117.2 | |
| B | 2.20 ± | 480.0 ± | 345.7 ± | 1373.4 ± | 1.03 ± 0.23 |
| | 0.796 | 0.0 | 139.2 | 730.3 | |
| C | 2.15 ± | 278.3 ± | 336.8 ± | 1654.0 ± | 1.00 ± 0.00 |
| | 0.669 | 307.5 | 121.2 | 1354.8 | |
| D | 1.94 ± | 419.8 ± | 305.3 ± | 1391.1 ± | 0.94 ± 0.27 |
| | 0.850 | 206.3 | 109.8 | 395.7 | |

1. A process for the preparation of a spray-dried composition, the composition comprising i) talnetant particles having a D_{V}90 in the range from 0.1 to 2.0 μm, ii) one or more ionic surfactant and iii) one or more soluble carrier, the process comprising a) wet milling a dispersion of the solid talnetant particles until the D_{V}90 is in the range from 0.1 to 2.0 μm, which dispersion comprises the one or more ionic surfactant and the one or more soluble carrier, then b) spray drying or spray granulating the resulting dispersion.

2. A process according to claim 1 wherein the dispersion is wet-milled in a water-based medium.

3. A process according to claim 1 wherein the dispersion contains 5 to 50% w/w of talnetant.

4. A process according to preceding claim claim 1 wherein the dispersion contains 15 to 30% w/w of talnetant.

5. A process according to claim 1 wherein the ionic surfactant is an anionic surfactant.

6. A process according to claim 1 wherein the ionic surfactant is sodium lauryl sulfate or dioctyl sodium sulfosuccinate.

7. A process according to claim 1 wherein the ionic surfactant is sodium lauryl sulfate.

8. A process according to claim 1 wherein the concentration of surfactant in the spray dried composition is 0.5 to 3.0% by weight of talnetant.

9. A process according to claim 1 wherein the concentration of surfactant in the dispersion prior to spray drying is 0.05 to 5.0% by weight of dispersion.

10. A process according to claim 1 wherein the dispersion contains 0.001 to 0.1 moles of ionic surfactant per mole of talnetant.

11. A process according to claim 1 wherein the one or more soluble carrier is a soluble sugar.

12. A process according to claim 1 wherein the one or more soluble carrier is selected from the group consisting of mannitol, sorbitol, lactose, lactitol, xylitol, trehalose, dextrose, sucrose, maltose, fructose, maltilol, xylitol, erythritol, polydextrose, isomalt, cyclodextrin and starch.

13. A process according to claim 1 wherein the spray dried composition comprises one or more soluble carrier selected from the group consisting of mannitol, lactose, erythritol, polydextrose, isomalt and lactitol.

14. A process according to claim 1 wherein the concentration of the one or more soluble carrier in the spray dried composition is 10 to 75% by weight of talnetant.

15. A process according to claim 1 wherein the concentration of the one or more soluble carrier in the dispersion prior to wet milling or after wet milling is 0.1 to 30% by weight of dispersion.

16. A process according to claim 1 wherein the spray-dried composition comprises one or more anti-agglomeration agents.

17. A process according to claim 1 wherein the concentration of the anti-aglomeration agent in the spray-dried composition is 2 to 10% by weight of talnetant.

18. A process according to claim 1 wherein the concentration of anti-aglomeration agent in the dispersion prior to spray drying is 0.1 to 10.0% by weight of dispersion.

19. A spray dried pharmaceutical composition comprising i) talnetant particles having a D_{V}90 in the range from 0.1 to 2.0 μm, ii) one or more ionic surfactant and iii) one or more soluble carrier.

20. A pharmaceutical composition according to claim 19 wherein the ionic surfactant is an anionic surfactant.

21. A pharmaceutical composition according to claim 19 wherein the ionic surfactant is sodium lauryl sulfate or dioctyl sodium sulfosuccinate.

22. A pharmaceutical composition according claim 19 wherein the ionic surfactant is sodium lauryl sulfate.

23. A pharmaceutical composition according to claim 19 wherein the concentration of surfactant in the spray dried composition is 0.5 to 3.0% by weight of talnetant.

24. A pharmaceutical composition according to claim 19 wherein the one or more soluble carrier is a soluble sugar.

25. A pharmaceutical composition according to claim 19 wherein the one or more soluble carrier is selected from the group consisting of mannitol, sorbitol, lactose, lactitol, xylitol, trehalose, dextrose, sucrose, maltose, fructose, maltilol, xylitol, erythritol, polydextrose, isomalt, cyclodextrin and starch.

26. A pharmaceutical composition according to claim 19 wherein the spray dried composition comprises one or more soluble carrier selected from the group consisting of mannitol, lactose, erythritol, polydextrose, isomalt and lactitol.

27. A pharmaceutical composition according to claim 19 wherein the concentration of the one or more soluble carrier in the spray dried composition is 10 to 75% by weight of talnetant.

28. A pharmaceutical composition according to any claim 19 wherein the spray-dried composition comprises one or more anti-agglomeration agents.

29. A pharmaceutical composition according to any claim 19 wherein the concentration of the anti-aglomeration agent in the spray-dried composition is 2 to 10% by weight of talnetant.

30. A dosage form comprising a composition defined in claim 19.

31. A dosage form according to claim 30 administered orally.

32. A dosage form according to claim 31 administered as a tablet.
